(19) United States
(12) Patent Application Publication
(10) Pub. No.: US 2013/0316430 A1
(43) Pub. Date: Nov. 28, 2013

(54) POLYVINYL CHLORIDE SURFACE CO-IMMOBILIZED WITH ENZYMES AND USES THEREOF

(71) Applicants: Chandra Shekhar Pundir, Haryana (IN); Nidhi Chauhan, Haryana (IN)

(72) Inventors: Chandra Shekhar Pundir, Haryana (IN); Nidhi Chauhan, Haryana (IN)

(73) Assignee: MAHARSHI DAYANAND UNIVERSITY, Haryana (IN)

(21) Appl. No.: 13/983,294

(22) PCT Filed: Dec. 7, 2011
(86) PCT No.: PCT/IN2011/000833

§ 371 Date: Aug. 1, 2013

(30) Foreign Application Priority Data
Feb. 1, 2011 (IN) 234/DEL/2011

(51) Int. Cl. C12N 11/18

(52) U.S. Cl. CPC C12N 11/18
USPC 435/175

(57) ABSTRACT

A PVC surface co-immobilized with the multiple enzymes for removal of stains useful in the field of washing or cleaning cloth and other household textile such as towels and sheets. The present invention also provides a process of preparation of the PVC surfaces and using such PVC surface. The PVC surface co-immobilized with the enzymes is useful as cheap and reusable alternative for washing of cloths.

### FIELD OF INVENTION

The present invention relates to enzyme co-immobilized polyvinyl chloride surface and various uses thereof.

### BACKGROUND OF THE INVENTION

Enzymes have been used in laundry products as cleaning and fabric care agents. Most of the enzyme used breakdown the large, water-insoluble soil and stains attached to fabrics/chinaware into smaller, more water-soluble pieces. Subsequently, the smaller molecules are removed, by the mechanical action of the (dish) washing machine or by the interaction of other detergent ingredients. The enzyme does not loose its functionality alter having worked on one stain and continues to work, on the next one. Enzymes also deliver fabric care benefits by better maintaining whiteness or keeping colors bright. The most important reasons to use enzymes in detergents are i) Very small quantity of these inexhaustible bio catalysts replace very large quantity of man made chemicals and ii) enzymes can work at very low temperature at which traditional chemicals are quite often no longer effective iii) they are fully biodegradable.

The following classes of enzymes are known to improve the laundry process: Proteases act on soils and stains containing proteins e.g. collar & cuff soil-lines, grass and blood. Proteases are enzymes that break down a long protein into smaller chains called peptides (a peptide is simply a short amino acid chain). Amylases, remove starch-based soils and stains. e.g. sauces, ice-creams, gravy. Amylases break down starch chains into smaller sugar molecules. Lipases are effective in removing oil/greasy body and food stains. Cellulase provide general cleaning benefits, especially on dust and mud and also work on garments made from cellulosic fibers, minimizing pilling to restore color and softness.

The costly detergent powder being sold in the market is a mixture of chemical detergent and enzymes, which constitute 90 percent and 10 percent of the powder respectively. The enzymes are being mixed in the detergents for easy removal of stains from clothes. Hence, the cost of the detergent is higher than that of the ordinary detergents. However, there is a drawback of these enzymatic detergents that it provides the single use of enzymes.

α-Amylase has been used in commercial detergents for washing or removal of starch stain from clothes (Rani, P., et al., 2007, Indian L Biotech. 6:230-233). Surface hydrophobic amino acid residues in cellulase molecule as structural factor responsible for their washing performance; remove the stain of cellulosic fibers from clothes. Hence, cellulases have found a new significant role in detergent industries. Cellulases used currently in detergents are not generally stable and hence required to be protected from the proteases attack and other components of detergents such as surfactants, which otherwise inhibit cellulose activity.

α-Amylase from different sources has been immobilized on supports such as, human erythrocytes, cellulose, polystyrene, alkylamine glass beads through covalent coupling, cation exchange resin, photographic gelatin, plastic supports, agar gel, acrylonitrile/acrylamide membranes, poly(2-hydroxyethyl methacrylate) microspheres, poly (methyl methacrylate-acrylic acid) microspheres, polyacrylamide gel, glass beads, sodium alginate beads, superporous celbeads, polyster surface free and affixed alkyl and arylamine glass beads, alginate gel beads, cyclic carbonate bearing hybrid materials, celluiose fibre materials and cellulose-coated magnetite (CCM) nanoparticles.

Among the various supports employed for immobilization of cellulase were polyurethane foam, tri(4-formyl phenoxy) cyanurate, polyacrylamide-acrylic gel, acrylamide grafted acrylonitrile copolymer (PAN), chemically modified pumic particles, nanofibrous poly (vinyl alcohol) PVA, passive epoxy acrylate films modified by magnetic filtered plasma stream, silicate clay mineral, modified polyvinyl alcohol coatefl chitosan beads, loofa sponge, liposomes, brick dust via glutaraldehyde and Silicon wafers or amino terminated surface.

The proteases from different sources have been immobilized onto various supports by different methods for different purposes such as protease from animal pancreas onto surface of the copoly (ethylene/acrylic acid) fiber through covalent fixation and an ionic interaction, protease, flavourzyme onto Lewatit R-258-K by covalent binding using glutaraldehyde, activated thiol-Sepharose, immobilization and stabilization of the enzyme from papaya latex onto chelating Sepharose, papain on macro porous polymer carrier by glutaraldehyde cross linking and diazo-coupling, protease from Bacillus licheniformis onto physicochemical characterized silica supports by covalent coupling, protease from fungi onto gold nanoparticle loaded zeolite microsphere, protease on various matrix by entrapment method, on nano particle surface, on cotton gauze bandage, highly porous activated carbon (HPAC), on functionalized mesoporous activated carbon (FMAC) and on the TiO₂ nanoparticles assembled on the porous MCM-41 (Mobile Crystalline Material No. 41) particles.

The lipases from different sources have been immobilized on various supports by different methods for different purposes such as lipase from Candida rugosa onto Sepharose 6-B through covalent coupling, lipase from Pseudomonas fluorescence on to polystyrene butadiene rubber using two phase emulsion technique for the hydrolysis of triacetin, lipase from Rhizopus onto polyvinyl chloride ultra-filtration membranes using phase-inversion process for plant oil hydrolysis, lipase from Candida rugosa onto micro-porous polypropylene by adsorption, lipase from Candida cylindracea, Aspergillus niger and Pseudomonas fluorescence onto anion exchange resin and diatomaceous-earth through adsorption for its use in trans-esterification and esterification reactions is non-aqueous system, lipase from Candida rugosa onto porous polyurethane particles, and organic polymer beads to increase the stability of enzyme against detergent and onto polyethylene powder for its use in the production of alpha-linolenic acid from perilla-oil hydrolysis, different lipases on EP-100 polypropylene powder by adsorption phenomenon, lipase from Pseudomonas cepacia within a phyllosilicate sol-gel matrix, Pseudomonas sp. lipase on a hollow tube reactor by covalent coupling for the multi-response kinetics of hydrolysis of corn oil through enzyme reactor, lipase from Candida antarctica on acrylic resin for the synthesis of ascorbate fatty acid esters in organic media, lipase from porcine pancreas on to free alkylamine glass beads through glutaraldehyde coupling and its use in washing of cotton clothes in presence of various detergents, Chromobacterium viscosum lipase onto solid nano composite matrix of gelatin hardened by polymerization of tetramethoxysilane (TMOS) through entrapment method, Pseudomonas cepacia lipase on phyllosilicate sol-gel matrix through entrapment technique for the production of alkylestors of restaurant grease as bio-diesel, lipase from Bacillus GK8 on different supports like silica, sepabeads, CNBr activated Sepharose 4B, HP-20 beads and phenyl-sepharose through glutraldehyde covalent coupling, lipase on to alkylamine glass beads affixed inside a plastic beaker through glutaraldehyde coupling and its use in washing of cotton cloth in presence of various detergents. Candida rugosa lipase on γ-Fe₂O₃ magnetic nano particles through covalent coupling for long term stability and calcium alginate gel, in inorganic microcapsules of calcium silicate and on macroporous acrylic beads to enhance the thermostability of lipase and onto natural kaolin by physical adsorption method, Candida sp. 99-125 lipase onto macroporous resin to display the highest activity in synthesis of biodiesel in Low Aqueous Media, Pseudomonas cepacia lipase on celite for the preparation of biodiesel from Jatropha oil in a solvent free system onto onion membrane affixed on plastic surface. Lipase from Penicillium simplicissimum on hydrophobic supports by selective adsorption.

Cholesterol esterase & cholesterol oxidase have also been co-immobilized covalently inside a plastic beaker, which served as electrochemical cell for three electrode-based system for measurement of serum total cholesterol (Hooda, V., et al., 2009, Sens. and Actuat. B. 136:235-241).

Lipase, trypsin and α-amylase have been co-immobilized onto the surface of non-woven polyester material to achieve a uniform distribution of the various enzyme species where the different enzyme activities were bound on the support (Bachmann, M. N., et al., 2007, Biotechnol Bioeng. 96:623-30).

Lipase along with glycerol kinase, glycerol-3-phosphate oxidase and peroxidase was co-immobilized onto free and affixed arylamine and alkylamine glass beads (Kalia, V., et al., 2002. Indian J. Biochem. Biophys. 39:342-346) affixed on plastic strip through diazotization for determination of triglycerides in serum (Kalia, V., et al., 2002, Indian J. Biochem. Biophys. 39:342-346, Minakshi, et al., 2008, Indian J of Biochem. Biophy. 45:111-115).

The utility of immobilized α-amylase in removal of starch stain from cotton cloth by various detergents was tested by chemical method. All the detergents gave better washing in presence of immobilized α-amylase than that by detergent alone (Rani, P., et al. 2007, Indian J. Biotech. 6:230-233).

Lipase along with glycerol kinase (GK), glycerol-3-phosphate oxidase (GPO), and horseradish peroxidase (HRP) has been co-immobilized onto artificially prepared PVC membrane for its use in amperometric determination of triglyceride (Narang, J., et al 2010, Anal. Lett. 43:1-11).

However α-amylase, cellulose, protease and lipase have not been co-immobilized so far. We report herein for the first time, the covalent co-immobilization of commercial α-amylase, cellulase, protease (purified from soybean seeds) and lipase onto plastic beaker and brush and their repeat use in cloth washing by non enzymic detergent. Polyvinyl chloride (PVC) sheets is a promising material for enzyme immobilization owing to the PVCs properties such as being chemically inert, corrosion free, weather resistant, tough, lightweight and maintenance free and having ease in molding to various shapes & size due to high strength-to-weight ratio (Pundir, C. S., et al., 2008, Anal. Biochem. 374:272-277).

U.S. Pat. No. 7,250,253 describes the invention related to polyfunctional polymer monolayers (polymer brushes) comprised a multitude of polymer chains attached to a surface, with each polymer chain comprised a multitude of units carrying at least on functional group which allows the interaction of the polymer chain with a sample molecule.

U.S. Pat. No. 3,860,536 describes a stable aqueous formulation of an enzyme detergent combination. The stability of this aqueous formulation permits a wider applicability in laundering textile fabrics.

U.S. Pat. No. 5,707,950 discloses the invention concerned laundry detergent compositions comprising lipase (especially the variant D96L of the native lipase derived from Humicola lanuginosa), protease and surfactant, wherein said compositions comprised levels of lipase and protease such that the whitening performance of said compositions was increased. These compositions deliver an improved whiteness maintenance and/or dingy clean-up on fabrics.

U.S. Pat. No. 3,637,339 describes composition for removing stains from fabrics, including, an enzyme, a per-compound and an activator for the perborate. A process for removing stains from fabrics which comprised subjecting said fabrics to aqueous wash water to which the following composition had been added in amount sufficient to provide about 1 to 40 p.p.m. of available oxygen, said composition consisting essentially of effective amounts of an inorganic peroxygen compound, an activator for the peroxygen compound, said activator being present in amount effective to so convert said peroxygen compound and thereby increase the bleaching effect of said peroxygen compound on coffee/tea stains.

U.S. Pat. No. 7,469,703 provides a motorized stain-removal brush. A method of using motorized stain-removal brush for cleaning inanimate surfaces was also provided. The motorized stain-removal brush includes a handle having—a motor disposed therein, a head having a longitudinal axis, and a neck disposed between the handle and the head. Bristle holders were associated with the head. The motor was operatively connected to the bristle holder.

U.S. Pat. No. 5,858,117 discloses compositions for use as soil removing agents in the food processing industry. Food soiled surfaces in food manufacturing and preparation areas can be cleaned. The compositions were manufactured in the form of a concentrate which was diluted with water and used. The cleaning materials were made in a two part system which were diluted with a diluent source and mixed prior to use. The products contain high quality cleaning compositions and use a variety of active ingredients. The preferred materials, in a two part system contained detergent compositions, enzymes that degrade food compositions, surfactants, low alkaline builders, water conditioning (softening) agents and optionally a variety of formulary adjuvant depending on product form.

U.S. Pat. No. 6,265,191 describes immobilization of lipase on surfaces to facilitate oil removal from the surfaces and to alter wet ability of the surfaces. The lipase was isolatable from a Pseudomonas organism such as Pseudomonas putida ATCC 53552 or from an organism expressing a coding region found in or cloned from the Pseudomonas. A particularly preferred lipase had a molecular weight of about 30 to 35 KD and was resolvable as a single band by SDS gel electrophoresis. Lipase absorbed on fabric forms a fabric-lipase complex for oil stain removal.

U.S. Pat. No. 5,232,843 describes lipase supported on a carrier material, which might hydrophobic or formed of an ion-exchange resin, by adsorbing to the carrier the lipase and a substantial coating of a non-lipase protein such as ovalbumin, bovine serum albumin or sodium caseinate. The protein was applied simultaneous with or prior to the lipase. The protein coating improved the activity of the enzyme especially with respect to its use in esterification and inter-esterification reactions.

U.S. Pat. No. 6,596,520 describes immobilized lipase prepared by adsorbing lipase from a crude lipase solution onto polyolefin particles such as polypropylene particles which were nonpolar. The crude solution might be a cell-free culture broth. Lipase sources included Pseudomonas burkholderia and Pseudomonas aeruginosa. Used of the immobilized lipase include enantioselective conversion of substrates such as enantioselective acylating or hydrolyzing.

U.S. Pat. No. 5,445,955 disclose an immobilized lipase prepared for trans-esterification of oils, fats or phospholipids in a reaction system containing a very small amount of water such as 50 to 2,000 ppm. The lipase might be a phospholipase such as phospholipase A2. In a first embodiment, lipase from a microorganism of the genus Rhizopus, Mucor, Alcaligenes or Candida was immobilized on the surface of a hydrophobic, insoluble organic polymer carrier having pores of an average diameter of 10 nm or larger. A solution of lipase was contacted with the polymer carrier for 10 minutes to 40 hours to covalently bond the lipase to the carrier. The immobilized lipase was dried under reduced pressure to a water content of 0.5 to 30 wt %.

U.S. Pat. No. 4,343,901 discloses a magnetic support matrix for enzyme immobilization was prepared which comprises a porous, refractory inorganic oxide containing ferromagnetic particles dispersed throughout its interior and a polyamine cross-linked with an excess of a bifunctional reagent impregnated therein so as to furnish pendant functional groups. Such a magnetic support matrix did not otherwise substantially decrease loading of subsequently immobilized enzyme, nor in any other way substantially alter the properties of the immobilized enzyme system when compared to that prepared on a non-magnetic support.

U.S. Pat. No. 5,474,925 discloses transgenic cotton plants had been created which expressed an immobilized protein in the cotton fiber cells. The cotton fiber could be recovered from such transgenic cotton plants and then used as a substrate for fixing immobilized protein for use in industrial or laboratory processes. US researchers had shown that they could make plastic films containing active enzymes.

US Patent application 20060053567 discloses an applicator for a fabric treatment composition and its application. More specifically the invention related to a versatile, effective convenient to apply fabric treatment applicator and its method of application. Claimed and described was method for the application of a fabric treatment composition, which comprises bleach and which was left to evaporate after being applied to a fabric.

US Patent application 20040161860 discloses multivariate type assay methods which comprises co-immobilizing different groups of ligands on respective discrete areas of a solid support surface, sequentially contacting the different areas with single or multiple analytes, detecting interactions of the analytes with the ligand groups, and determining there from the amount of ligand-binding of each analyte.

The process was based on one, typically used to produce thin, flat plastic products such as CDs, DVDs and flat-screen displays (University of Minnesota, St. Paul, US). They modified thin film with polystyrene chemically to bind with enzymes (Tong et al. 2008 Biotechnol. Progr. 24:714-719)

University of Minnesota Researcher Ping Wang used a disc measuring 10 centimeter across, which he coated in a layer of four films (Tong et al., 2008 Biotechnol. Progr. 24:714 719). “The bonding between the enzyme and polymer coating was as strong as the chemical bonds that were responsible for the integrity of plastics”, said Wang while explaining that harsh chemical treating could not destroy the enzyme protection. In spite of that, the enzymes were active enough to dissolve proteins as soon as stains occur.

### SUMMARY OF THE INVENTION

One aspect of the present invention provides a polyvinyl chloride surface for removing stains, wherein the polyvinyl chloride surface is co-immobilized with enzymes lipase, cellulase, amylase and protease.

This aspect and other features and advantages of the present invention will become better understood with reference to the following figures, description, examples and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING FIGURES

FIG. 1 shows scheme of covalent immobilization of enzymes onto PVC/plastic surface and its application in removal of stain (washing of cloths).

FIG. 2 shows scanning electron micrographs (SEM) of chemically modified PVC surface with (A) and without (B) co-immobilized enzymes.

FIG. 3 shows effect of pH on free (purified from soybean seeds) and co-immobilized protease onto plastic beaker/brush

FIG. 4 shows effect of incubation temperature on the activity of free & co-immobilized protease on plastic beaker/brush

FIG. 5 shows effect of time incubation on activity of free and co-immobilized protease onto plastic beaker/brush

FIG. 6 shows effect of substrate concentration on activity of free and co-immobilized protease onto plastic beaker/brush

FIG. 7 shows effect of pH on plastic beaker/brush bound α-amylase

FIG. 8 shows effect of incubation temperature on plastic beaker/brush bound α-amylase

FIG. 9 shows effect of time incubation on plastic beaker/brush bound α-amylase

FIG. 10 shows effect of substrate concentration on plastic beaker/brush bound amylase

FIG. 11 shows effect of pH on plastic beaker/brush bound cellulase

FIG. 12 shows effect of temperature on plastic beaker/brush bound cellulase

FIG. 13 shows effect of incubation time on plastic beaker/brush bound cellulase

FIG. 14 shows effect of substrate concentration on plastic beaker/brush bound cellulase

FIG. 15 shows effect of pH on plastic beaker/brush bound lipase

FIG. 16 shows effect of temperature on plastic beaker/brush bound lipase

FIG. 17 shows effect of incubation time on plastic beaker/brush bound lipase

FIG. 18 shows effect of substrate conc. on plastic beaker/brush bound lipase

FIG. 19 shows Lineweaver-Burk plots of free protease onto plastic beaker/brush

FIG. 20 shows Lineweaver-Burk plots of co-immobilized protease onto plastic beaker/brush

FIG. 21 shows Lineweaver-Burk plots of co-immobilized α-amylase onto plastic beaker/brush

FIG. 22 shows Lineweaver-Burk plots of co-immobilized cellulase onto plastic beaker/brush

FIG. 23 shows Lineweaver-Burk plots of co-immobilized lipase onto plastic beaker/brush

FIG. 24 shows effect of reusability on the activity of α-amylase, cellulase, protease and lipase respectively, immobilized on PVC sheet over a period of 100 days when stored at 4° C.

FIG. 25 shows reusability of co-immobilized enzymes on PVC sheet

### DETAILED DESCRIPTION OF THE INVENTION

The present invention provides polyvinyl chloride (PVC) surface co-immobilized with enzymes lipase, amylase, protease and cellulase. The present invention also provides a process of preparation of PVC surface co-immobilized with the enzymes and using such PVC surfaces. The PVC surface co-immobilized with enzymes as disclosed in the present invention is useful in the field of washing or cleaning cloth and other household textile such as towels and sheets.

The present invention particularly provides a PVC surface co-immobilized with enzymes lipase, amylase, protease and cellulose in the form of container and brush, wherein the enzymes are co-immobilized using a coupling agent having two terminal —CHO groups, of which one is attached to the PVC surface and other is attached to —NH₂ group on enzyme (protein) surface, through covalent coupling.

The inside of container and brush were treated with inorganic acids followed by treatment with a coupling agent having at least two terminal —CHO groups in aqueous buffer solution to obtain activated solid support which is further contacted with a enzymes having at-least a free amino functional group.

The co-immobilization of enzymes on a PVC surface not only provides reuse of enzymes but also protects it from protease action and surfactant inhibition. Immobilized enzymes are very important for detergents uses as they possess many benefits to the expenses and processes of the reaction of which include: Convenience: Minuscule amounts of protein dissolve in the reaction, so workup can be much easier. Upon completion, reaction mixtures typically contain only solvent and reaction products. Economical: The immobilized enzyme is easily removed from the reaction making it easy to recycle the biocatalyst. Stability: Immobilized enzymes typically have greater thermal and operational stability than the soluble form of the enzyme.

The PVC container and brush co-immobilised with enzymes lipase, amylase, protease and cellulase are useful as cheap and reusable alternative for washing of cloths.

Nevertheless, if the enzymes are used in immobilized form in chemical detergent, it was not only worked with more efficiency but also reused 200 times. The repeated use of the same quantity of enzyme in detergent would reduce the cost of washing. Enzymes are co-immobilised onto the inner wall of a plastic beaker and brush with the help of some specific chemicals, which provided the reuse of enzyme α-amylase, cellulase, protease and lipase are being used in its free form in detergent available in the market, for easy removal of stains of clothes. The enzyme in its free form can be used once. However it can be re-used 200 times, if it is immobilized.

Accordingly, the invention provides an enzymes co-immobilized container and brush comprising of α-amylase, cellulase, protease and lipase. The enzyme co-immobilization is useful for removal of stains from cloths.

There is also provided a process of preparation of an enzyme co-immobilized container and brush comprising of α-amylase, cellulase, protease and lipase.

In one embodiment the present invention provides a PVC surface co-immobilized with enzymes α-amylase, cellulase, protease and lipase, wherein the PVC surface is in the form of container or brush and is useful for stains removal from cloths. The co-immobilized enzymes onto container and brush are useful as cheap and reusable alternative for washing of cloths.

In accordance with the present invention there is provided a polyvinyl chloride surface for removing stains, wherein the polyvinyl chloride surface is co-immobilized with enzymes lipase, cellulase, amylase and protease.

In another embodiment of the present invention there is provided a polyvinyl chloride surface for removing stains, wherein the polyvinyl chloride surface is co-immobilized with enzymes lipase, cellulase, amylase and protease, wherein the surface is in the form of sheet, brush, vessel, pipe, reactor, chips, disc, strip and gauge.

In another embodiment of the present invention there is provided a polyvinyl chloride surface for removing stains, wherein the polyvinyl chloride surface is co-immobilized with enzymes lipase, cellulase, amylase and protease, wherein the PVC surface is capable of removing stains in distilled water, canal water, ground water, tap water, well water and hard water.

In another embodiment of the present invention there is provided a polyvinyl chloride surface for removing stains, wherein the polyvinyl chloride surface is co-immobilized with enzymes lipase, cellulase, amylase and protease, wherein the PVC surface is capable of is capable of removing stains in presence of non-enzymatic detergents.

In another embodiment of the present invention there is provided a polyvinyl chloride surface for removing stains, wherein the polyvinyl chloride surface is co-immobilized with enzymes lipase, cellulase, amylase and protease, wherein the PVC surface is capable of removing starch, protein, grass, oil, soil, blood, grease, sauces, ice-creams, gravies, egg, human sweat, chocolate, dust and mud.

In another embodiment of the present invention there is provided a polyvinyl chloride surface for removing stains, wherein the polyvinyl chloride surface is co-immobilized with enzymes lipase, cellulase, amylase and protease, wherein the PVC surface is capable of is reusable for at least 200 times.

In another embodiment of the present invention there is provided a polyvinyl chloride surface for removing stains, wherein the polyvinyl chloride surface is co-immobilized with enzymes lipase, cellulase, amylase and protease, wherein the PVC surface is capable of is polyvinyl chloride vessel or brush.

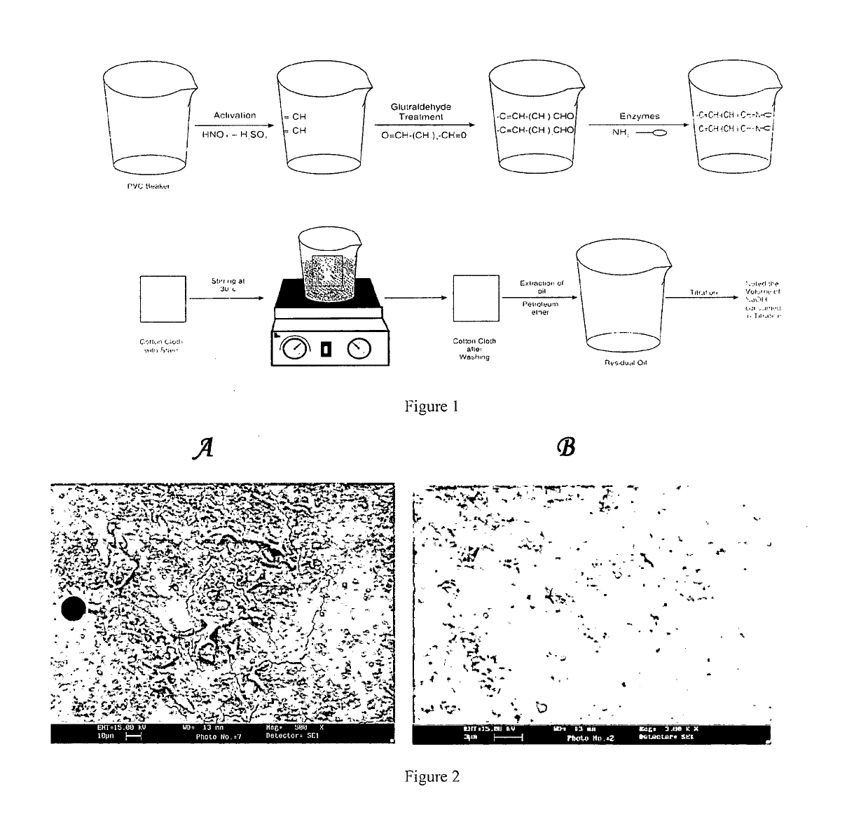

In another embodiment the present invention provides a process for producing the PVC surface co-immobilized with α-amylase, cellulase, lipase and protease, the process comprises co-immobilizing the enzymes purified from soybean seeds covalently onto inner surface of a plastic (PVC) beaker and brush with a conjugation yield of 0.02 mg/cm² and 0.016 mg/cm² respectively. The co-immobilized enzyme retained about 66.7, 54.2, 44.64, 62.8 (beaker bound) and 44.01, 66.23, 33.9, 45.8% (brush bound) of the initial activity of free enzyme respectively. During the immobilization, of. vinyl polymers of PVC material were broken down by treating with HNO₃/H₂SO₄ treatment which introduces nicks in long-chain polymer and generates free ends protruding from the polymer surface. This reaction of strong oxidizing agents removes chlorine molecules from damaged ends of polymer in a zipper action and introduces a double bond at the ends of these short-chain polymers (Pundir, C. S., et al., 2008. Anal. Biochem. 374:272-277). When free ends of treated polymer are reacted with the aldehyde group of a bifunctional cross-linking agent such as glutaraldehyde, one aldehyde group of a glutraldehyde reacts with free end of vinyl chain to form a —C═CH— bond between PVC sheet of glutraldehyde & thus leads to activation of surface for covalent immobilization of enzymes. The —NH₂ group on the surface of enzyme is attached covalently onto other —CHO group of glutraldehyde, already attached to PVC sheet through Schiff base formation of a Schiff. This leads to covalent linkage of protein/enzyme with PVC membrane surface (Pundir, C. S., et al., 2008, Anal. Biochem. 374:272-277) (FIG. 1).

### SEM of the PVC Sheets

The SEM of surface of chemically modified PVC sheets with the immobilized enzyme under high resolution resolved folds and clusters along some beaded structures which were not observed in membrane without enzyme (FIGS. 2 A and B, respectively). This change in surface morphology of the support after the immobilization confirmed the enzyme immobilization. The formation of folds instead of globular beaded structures may be due to a high concentration of co-immobilized enzymes on the surface of PVC membrane.

### Change in Kinetic Parameters

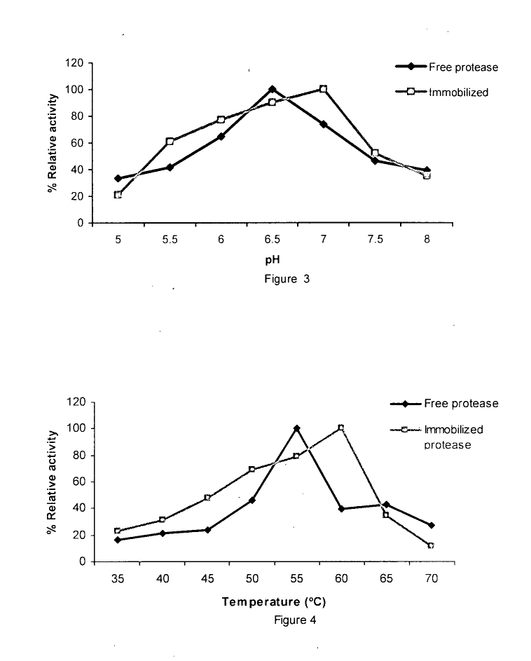

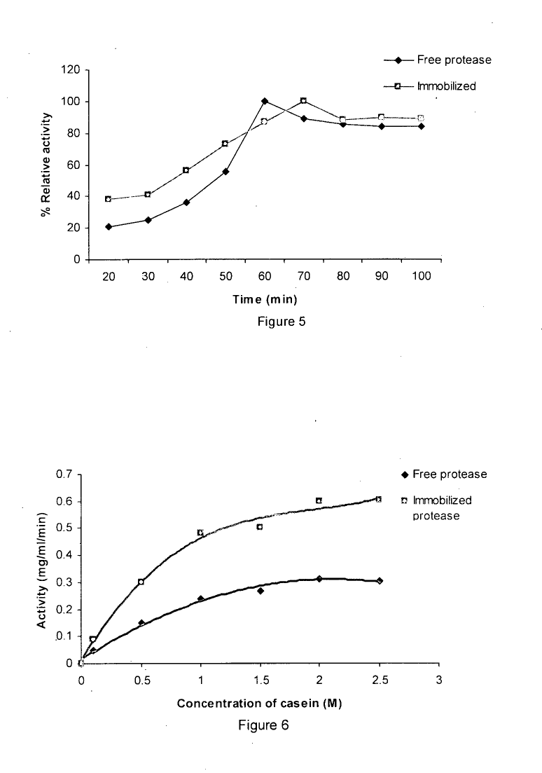

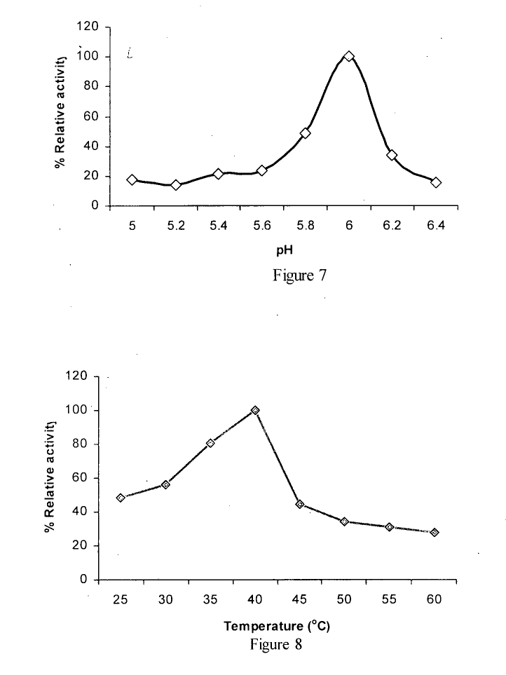

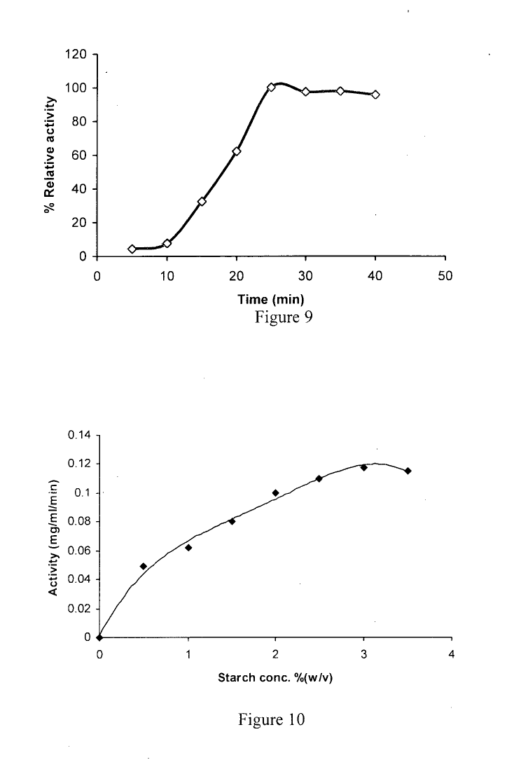

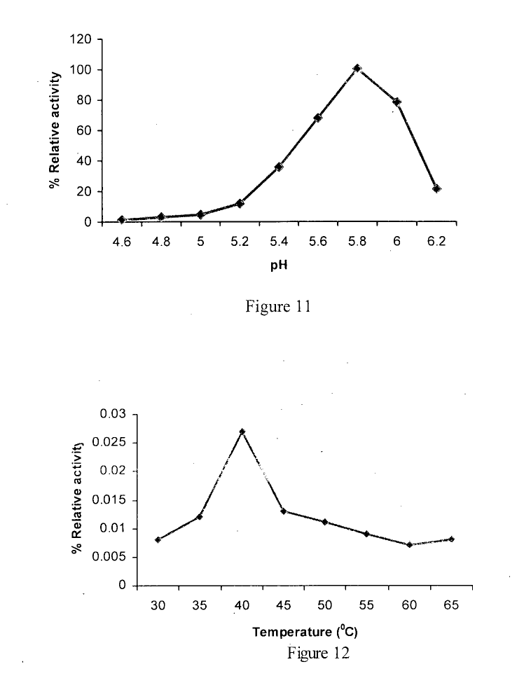

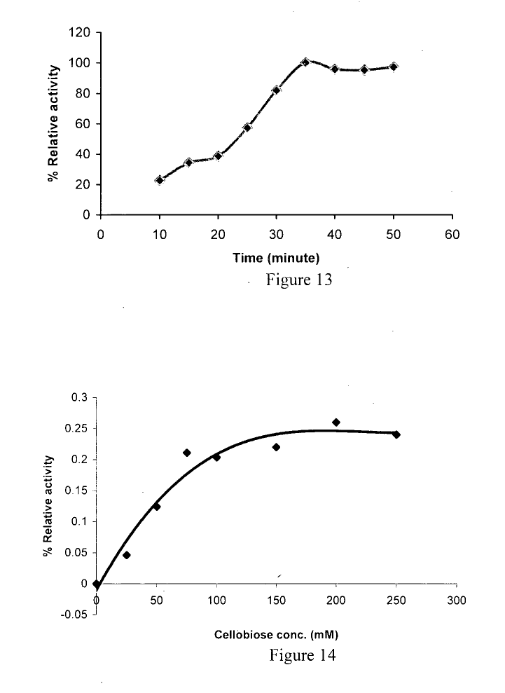

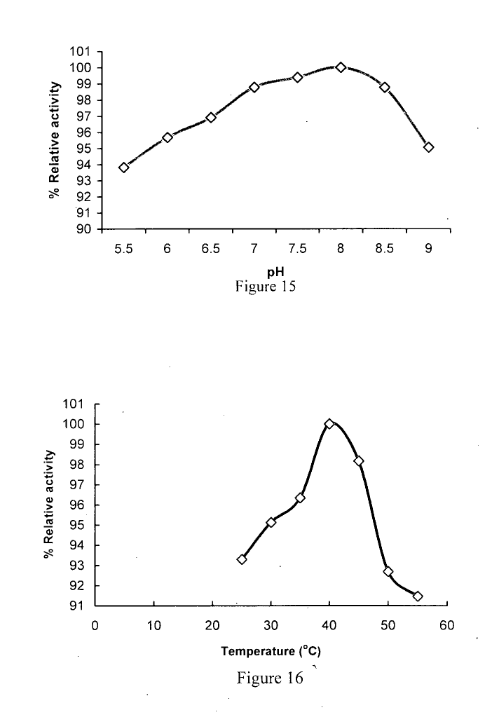

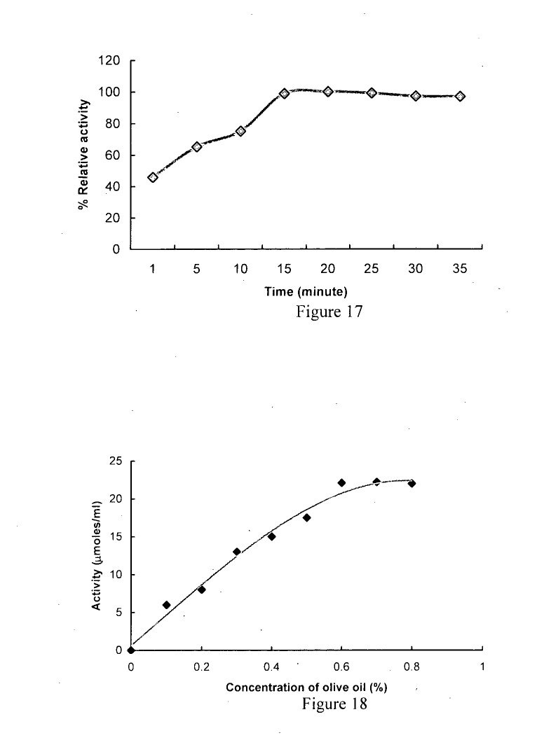

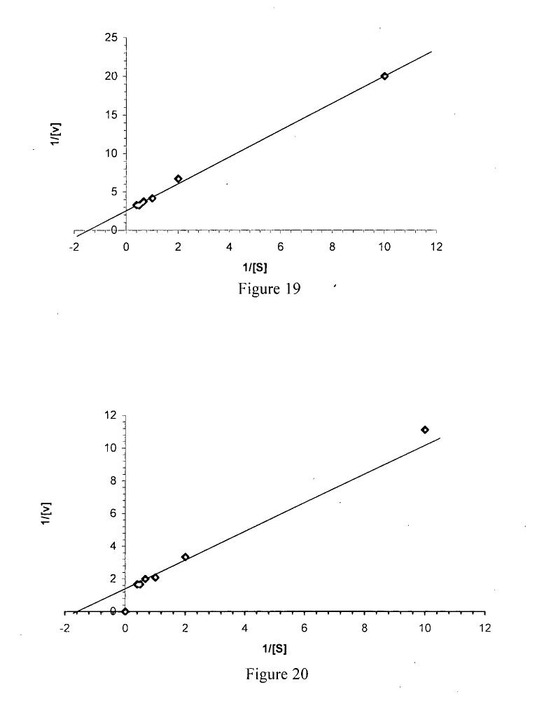

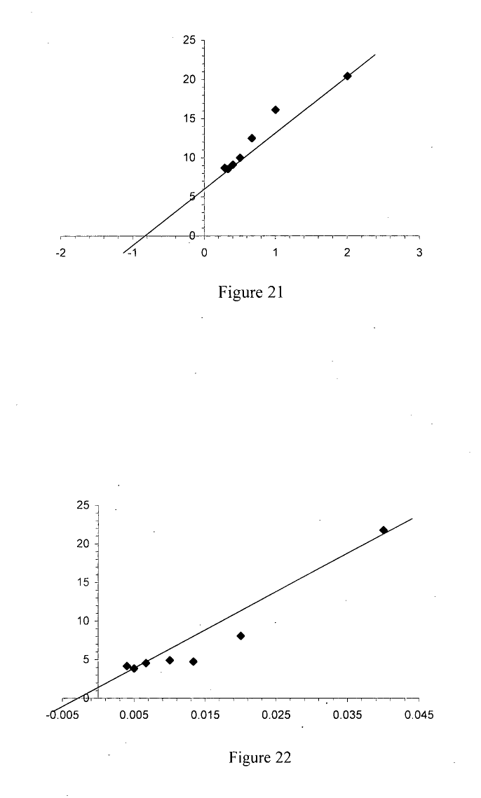

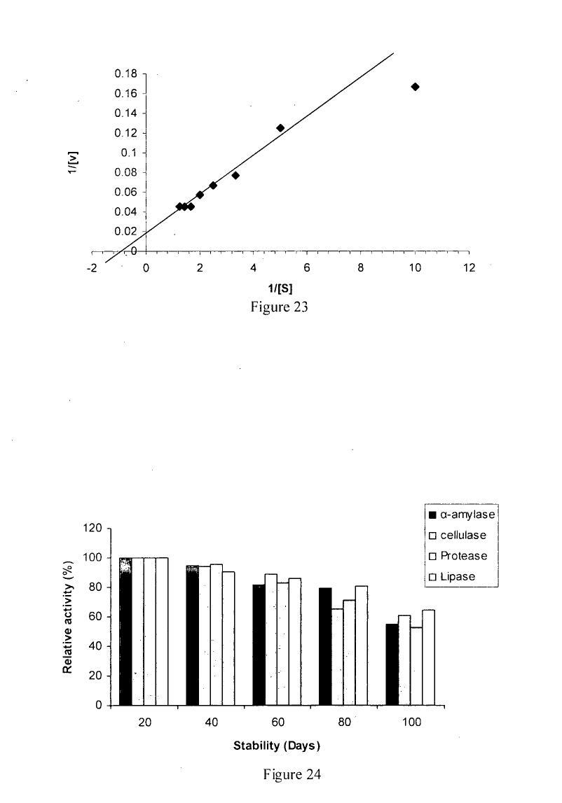

A comparison of various kinetic properties of co-immobilized enzymes with those of free enzymes is given in Table 1. The results showed that the enzymes under went minor changes after co-immobilization which revealed stability of enzyme and non-interference from PVC membrane support (FIGS. 3 to 23). FIGS. 3, 4, 5 and 6 shows the effect of pH, incubation temperature, time of incubation and substrate (casein) concentration on the activity of free and immobilized protease respectively. Standard assay conditions of protease were used except for the parameter on X-axis, which was varied as indicated. The optimum activity was considered as 100% and rest of the activities were calculated in % compared to optimum activity. Similarly FIGS. 7-10, 11-14 and 15-18 show the effect of these parameters on immobilized α-amylase, cellulase and lipase respectively. FIGS. 19 and 20 show the LB plot for free and co-immobilized protease, while FIGS. 21, 22 and 23 show the LB plot for co-immobilized α-amylase, cellulase and lipase onto plastic surface respectively.

Slight increase in optimum pH of enzyme after immobilization could be attributed to change in H⁺ concentration in enzymatic microenvironment due to loss of —NH₂ groups on the surface of enzyme during immobilization. The increase in optimum temperature of enzyme after immobilization might be due to slow transfer of heat from surrounding to catalytic system due to immobilization support barrier. Due to this reason, the temperature of system and surrounding varies and catalytic system needs slightly higher temperature of surrounding to maintain optimum catalytic temperature in system. Change in optimum temperature had also been observed in earlier immobilization studies (Kennedy, J. P., Hand Book of Enzyme Technology, Marcel Dekker Inc., New York, 1985). The change in Km and Vmax was also observed after immobilization. Km was slightly increased in case of α-amylase, cellulose and protease but decreased slightly in case of lipase. Rate of enzyme catalysis was measured in terms of Vmax. There was either slight decrease in Vmax or slight increase in case of α-amylase and cellulose. The change in Km and Vmax of an enzyme after immobilization depend upon many the change in microenvironment and product inhibition. Due to change in microenvironment of enzyme after immobilization, diffusibilities of substrate and products were different from that for native enzyme so change in Km and catalytic efficiency were generally observed (Pundir, C. S., et al., 2009, Talanta. 77:1688-1693).

Application of Co-Immobilization of Enzymes onto Beaker & Brush in Cloth Washing

The starch, grass, egg albumin and oil stained cotton cloth pieces were washed with detergents alone and in presence of co-immobilized enzymes. The content of residual of stain in cloth piece was determined as criteria of washing performance. The lesser the content of residual stain better the washing. Two types of detergents were tested, expensive (enzymic) detergents such as Surf Excel and non-enzymic detergents such as Ghari. This washing was carried out in four types of water i.e. Distilled water, canal water, ground water (hand pump water) and well water. The combination of any detergent+co-immobilized enzymes gave better washing than that by control (contained no detergent only water), enzymatic detergent & non-enzymatic detergent alone (Table-2 to 9). Table 2, 3, 4 and 5 shows comparison of washing performance (removal of starch, cellulose, egg albumin and oil stains from cotton cloth respectively) of non enzymatic detergent with enzymatic detergent in presence PVC/plastic beaker bound of co-immobilized α-amylase, cellulose, protease and lipase. The values given in the table represent the residual content of starch in cloth (mg/cm²) after washing. Table 6, 7, 8 and 9 shows the similar comparison using PVC/plastic brush bound enzymes.

Detergents normally contains surfactants, builders, co-builders, bleach, bleach activators and special additives, such as fluorescent brightener, filler, corrosive inhibitors, antifoaming agents and enzymes (in case of only enzymic detergents) and perfumes. Surfactants, major components of detergents are of four types: (i) anionic (e.g. sodium lauryl sulfate), (ii) cationic (e.g. hexadecyltrimethylammonium bromide as fabric softener), (iii) non-ionic (e.g. w-odecyloctaethylene glycomonoether ethoxylate), and (iv) amphoteric (e.g. laurylamido propyl dimethyl betaine as skin cleaner). A detergent may contain more than one type of surfactant. Hence, it is not possible to know the overall ionic state of a detergent, especially when the chemical composition of a commercial detergent is not available, due to professional secrecy. In the present case, the enzymes (α-amylase, cellulose, protease and lipase) were bound to PVC surface through covalent bonding and thus are affixed firmly on inner wall of plastic beaker & surface of brush. The enzyme in this form is hardly affected by the surfactant in solution. Hence, the comparison of washing performance of various detergents in the presence of various immobilized enzymes is due to their individual performance but not due to their different/combined effect on the enzyme. The co-immobilization of enzymes onto inert surface protects it from the effect of surfactant as well as provides its reuse.

### Storage Stability and Reusability

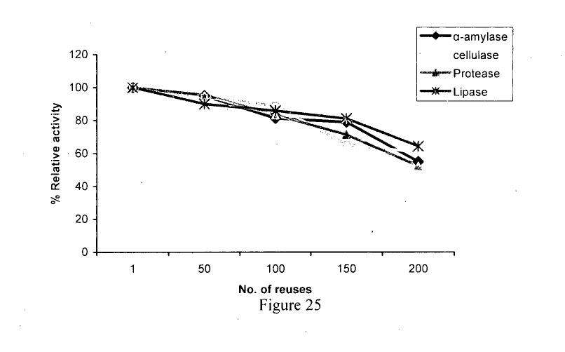

The co-immobilized enzymes were used for 200 times reused during the span of 3 months at 4° C. In such washings without any considerable loss of activity, when stored in cold (4-10° C.). Generally enzymes in free from is not safe as this might be attacked by proteases and inhibited by surfactant Thus, the use of beaker bound enzymes in washing of different stained cloths by detergents has not only increased their washing efficiencies without consuming them in the process but also made cheaper detergents equivalent to expansive detergents for washing purpose. The half life (⁺½) of co-immobilized enzyme was 3 months (FIGS. 24, 25). FIG. 24 shows the storage stability of α-amylase, cellulase, protease and lipase, co-immobilized on PVC sheet at 4° C. FIG. 25 displays the number of reuses co-immobilized enzymes over a period of 100 days, when stored at 4° C.

### EXAMPLES

The following examples are put forth so as to provide those of ordinary skill in the art with a complete invention and the description of how to make and use the present invention, and are not intended to limit the scope of what the inventors regard as their invention nor are they intended to represent that the experiments below are all and only experiments performed.

### Example 1

### Chemicals and Reagents

Cellulase from Trichoderma viridae, α-amylase (from bacterial source), lipase from porcine pancreas (40-70 U/mg protein), sodium potassium tartarate, dinitro salicyclic acid (DNS), anthrone TCA and starch were from SISCO Research Laboratory Pvt. Ltd., Mumbai. Glutaraldehyde (25%) was from Sigma St. Louis, USA. Acetone, methanol, ethanol and phenolphthalein were from E. Merck, Mumbai. Tris-base, calcium chloride and sodium benzoate were purchased from Qualigen mumbai, Mumbai. All other chemicals were of analytical reagent grade. White PVC beaker (100 ml) and brush, commercial enzymic detergents (Surf Excel) & non enzymatic detergents (Ghari), Olive-oil and seeds of soybean (Glycine max var. Ogden) were purchased from local market. Well, canal, ground (hand pump) water collected from nearby rural region of Rohtak.

### Example 2

### Extraction and Partial Purification of Protease from Soybean Seeds and Preparation of Crude Enzyme

### Extraction

Seeds of soybean were ground to a powder in a chilled waring blender with pauses every two min. Soybean flour (100 g) was mixed in 1.0 L of chilled distilled water in a chilled blender. This filled the container to capacity and minimized foam production. The suspension was blended for 6 min with pauses at 2 min intervals to prevent overheating. The suspension was blended for 6 min with pauses at 2 min intervals to prevent overheating. The resulting suspension was centrifuged at 15,000×g for 10 min at 4° C. A thin, white oily layer was skimmed off both the supernatant and pellet were collected and tested for enzyme activity and protein content (Weil, J., et al., 1966, Cereal chem. 3:392-399). The pellet was discarded as it showed very low activity and supernatant stored at 4° C. for further studies.

### Assay of Protease

The activity of protease was measured using the method of Nam Sun Wang with modifications. It was based on the quantification of amino acids produced from hydrolysis of casein by protease using a colour reaction of ninhydrin with amino acids. The enzyme was assayed in a 15 ml test tube. The reaction mixture contained 3.8 ml 0.05 M sodium phosphate buffer (pH 6.3) 0.1 ml 1% casein in reaction buffer and 0.2 ml of enzyme in a total volume of 4.0 ml. After incubation at 50° C. for 90 min, under continuous stirring, 0.5 ml ninhydrin (2% in acetone) was added to reaction mixture and was kept at 100° C. in boiling water bath for 10 min. Appearance of yellow colour showed the presence of amino acids. Turbidity, if any was settled down by centrifugation at 850×g for 10 min. A₅₇₀ nm recorded against control (run in similar manner as described above except that protease was replaced by reaction buffer). The concentration of the casein acid was determined from standard curve between casein concentration and A₅₇₀

### Determination of Protease

The protein content of various enzyme preparations was determined by the method of (Lowry, O. H., et al., 1951, J. of Biochem. 193:265-275) using bovine serum albumin (BSA) as standard protein.

### Purification of Protease

The crude enzyme was purified partially using the following purification techniques.

### TCA Precipitation

TCA was added to the supernatant (crude enzyme) to give a final concentration of 5%.

The mixture was stirred in cold until the TCA was dissolved. This mixture was put over night for precipitation. The solution was then centrifuged at 15000×g for 10 min. The precipitate was dissolved in minimum amount of reaction buffer and tested for proteolytic activity and protein.

### DEAE-Cellulose Column Chromatography and Preparation of DEAE Column

DEAE-cellulose was soaked in distilled water and allowed for swelling over night. The gel was treated with 2% HC 1 for 30 min and then washed in distilled water several times till the pH of washing discard was 7.0. Now the gel was treated with 2% NaOH for 30 min. Again the gel was washed several times with distilled water till the pH of washing discard was 7.0. A glass column (1.5×10) having glass wool at its lower end was fixed erect on a burette stand and its outlet was closed. The gel was stirred with the help of a glass rod and added slowly along the walls of the column with the help of a glass rod. The gel was allowed to settle for sometime. The column was then washed for 24 hr with 0.01 M sodium phosphate buffer pH 6.8 at the flow rate of 0.5 ml/min.

To load the sample first the input of eluent buffer was stopped and the eluent buffer was allowed to drain to the bed surface. The dissolved TCA precipitate was then placed on to gel gently along with the side wall of the column. The acetate buffer pH 4.8 was allowed to percolate through the column. This treatment solubilised part of the precipitate. The column was allowed to run in 0.01 M sodium acetate buffer pH 5.6 at a flow rate of 0.5 ml/min. Fractions of 3 ml then collected and tested for activity and protein.

### Example 3

### Free Enzymes Assay

α-Amylase assay

α-Amylase assay was based on measurement of glucose and maltose generated from hydrolysis of starch by α-amylase using DNS reaction. To 1.9 ml 0.05 M acetate buffer (pH −5.6) containing 2% starch in a test tube, 0.1 ml of enzyme solution was added. For blank, 2 ml reaction buffer containing 2% starch was taken in a test tube. Both blank and assay tubes were incubated at 37° C. under continuous stirring in a water bath. After incubation for 10 min, 0.1 ml 2N NaOH and 0.9 ml dinitro salicylic acid (DNS) reagent was added to both the test tubes. The test tubes were placed in boiling water bath for 5 min, cooled to room temperature and A₅₄₀ of red colour was read and the amount of glucose generated in reaction was extrapolated from standard curve between glucose concentration and A₅₄₀.

### Cellulase Assay

The assay of cellulase was based on the measurement of glucose generated from hydrolysis of cellobiose by cellulase using DNS reaction. To 1.9 ml of 0.05 M sodium phosphate buffer pH 7.0 containing 4.0 mg cellobiose in a test tube was added 0.1 ml dissolved enzyme in reaction buffer (1 mg/ml). For blank 2.0 ml of reaction buffer containing 4.0 mg cellobiose was taken. Both assay and blank tubes were incubated at 40° C. for 30 min in a water bath. After incubation, 0.1 ml 2N NaOH and 0.9 ml DNS reagent were added to both the tubes. The tubes were placed in boiling water bath for 10 min, cooled to room temperature and A₅₄₀ of red colour was read against blank and amount of glucose generated in the assay was extrapolated from standard curve between glucose conc. vs A₅₄₀.

### Lipase Assay

The activity of lipase was assayed according to Naher (Naher, G., 1974, Lipase titrimetric assay. In: Methods of Enzymatic Analysis, vol. 2. (Bergmeyer, H. IL, A©d.), pp. 814-818, Academic Press, New York) with modifications. In a 100 ml conical flask, 5.0 ml olive oil emulsion was added to 5.0 ml. 0.1 M tris buffer (pH 8.0) and incubated at 35° C. for 10 min. 1.0 ml lipase solution (5 mg/ml) was added and incubated at 35° C. for 20 min. The reaction mixture was then kept at room temperature for 20 min. Ten ml acetone and methanol mixture (1:1) was added to stop the reaction and titrated it against the 0.025N NaOH after adding 1% phenolphthalein as an indicator. Control was run for each sample to correct any drop in pH due to any factor other than lipase or incomplete termination of reaction by acetone and methanol mixture. In case of control, 1.0 ml of lipase solution was kept in boiling water bath for 5 min to get it heat denatured. Rest of the procedure was same as described for the test.

### Example 4

### Co-Immobilization of α-Amylase, Cellulase, Protease and Lipase onto PVC/Plastic Beaker & Brush

α-Amylase, cellulase, protease and lipase (1 mg/ml) in a mixture were co-immobilized onto the inner side of a PVC/plastic beaker and brush through covalent coupling using the method of Pundir et al 2008. The PVC surface was first incubated with nitrating acid (mixture of cone, nitric acid and sulfuric acid in 5:1 ratio) for 6 h to cleave polyvinyl chloride polymers oxidatively into small chain polymers having protruding ends toward the surface. This acid-treated PVC sheet was rinsed with water and incubated with 2.5% (w/v) glutaraldehyde solution in 0.05 M sodium phosphate buffer (pH 7.0) for 7 h at 30±5° C. The glutaraldehyde-treated surface was washed with distilled water many times or twice to remove excess of glutaraldehyde. The glutraldehyde activated PVC surface was incubated with solution of enzymes (Total 50 mg protein) in 50 mM sodium phosphate buffer (pH 7.0) at 4° C. for 24 h in the dark. The excess of enzyme was decanted off and tested for activity and protein concentration. The PVC beaker & brush were tested for activity of four enzymes.

### Example 5

### Scanning Electron Microscopy (SEM)

The PVC beaker and brush bound enzymes were subjected to scanning electron microscopy (SEM) to confirm the co-immobilization. In SEM, electrons reflect off the surface of a specimen coated with an evaporated gold-carbon film and are then collected by detectors for processing to produce a 3-dimensional-like image. The scanning electron microscopy of untreated and enzymes bound plastic base of beaker was performed at Electron Microscopy Facility, AIIMS, N. Delhi.

### Example 6

### Assay of Co-Immobilized Enzymes onto on PVC (Beaker/Brush)

The assay of co-immobilized enzymes was carried out in the same plastic beaker in which these were immobilized. The beaker was termed as “reaction beaker” and for the brush, it was carried out in a 100 ml flask containing plastic brush on which enzymes were co-immobilized. The assay procedure of co-immobilized enzyme was done in the similar manner as described for their free form except free enzyme was replaced by reaction buffer and reaction mixture was kept under constant stirring during incubation. After incubation, the reaction mixture was transferred to a test tube or flask. In case of plastic brush, the brush was taken off from the reaction mixture after incubation

### Kinetic Properties of Co-Immobilized Enzymes on PVC (Beaker/Brush)

The following kinetic parameters of co-immobilized enzymes were studied and compared with kinetic parameters of free enzyme: optimum pH, temperature, incubation period and effect of substrate concentration and calculation of Km and Vmax. To determine optimum pH of co-immobilized enzymes, the pH of reaction buffer was varied from 4.0 to 9.0 using different buffer systems within their effective pH ranges, e.g. 0.05 M sodium acetate for pH 4.0 to 6.0, 0.05 M sodium phosphate for pH 6.0 to 7.5, and 0.05 M Tris-HCl for pH 7.5 to 9.0. Similarly, for optimum temperature of co-immobilized enzymes, the reaction mixture was incubated at different temperatures ranging from 25 to 70° C. at interval of 5° C. The optimum time of co-immobilized enzymes was studied from 5-100 min at an interval of 5 min. To study the effect of substrate concentration on the initial velocity of co-immobilized enzymes, assays were performed at different concentration ranging from 0.1-3.5% starch for α-amylase, 25-250 mM cellobiose for cellulase, 0.1 to 3.5% casein for protease, and 30-100% olive oil for lipase. Km and Vmax values for co-immobilized enzymes were calculated from Lineweaver-Burk plot between reciprocal of substrate concentration [1/S] and reciprocal of initial velocity of the reaction [1/v].

### Example 7

### Applications of Co-Immobilized Enzymes in Cloth Washing

### Washing Performance of Co-Immobilized Enzyme

The rectangular pieces of white cotton cloth (Size: 4.5×4.5 cm) were used for the test and therefore termed as test clothes. These cloth pieces were stained with 0.2 ml of 2% starch, grass stain, 2% egg albumin and 0.2 ml mustard oil individually. The co-immobilized a-amylase, cellulose, protease and lipase were used to remove starch, grass, egg albumin and oil stain respectively. The commercial enzymic and non-enzymic detergent powders were dissolved in different water (Distilled water, canal water, ground water (hand pump) and well water) at a concentration of 2 g/L individually. 50 ml of detergent solution was transferred to plastic beaker. For each washing performance, 4 test cloth pieces were taken. 1 piece was washed with water alone. Second piece was washed with non enzymatic detergent third piece was washed with enzymatic detergent and fourth piece was washed with non enzymatic detergent in reaction beaker containing co-immobilized enzymes. In case of washing by beaker, the cloth was dipped into reaction beaker containing 50 ml detergent solution and in case of washing by brush; stained cloth was dipped into detergent solution for 2 min. and then rubbed 3-4 times with the help of brush. The washing was done at 35° C. for 20 min under continuous shaking, after which it was rinsed 2 times manually with water. Four types of water were used in each washing: Distilled water, canal water, ground water (hand pump) and Well water collected from nearly rural region of Rohtak city. The washing performance was judged by qualifying the residual stain after washing (starch/cellulose/protein/oil).

Determination of Residual Starch Content in Test Clothes after Washing

The cloth was dipped into 5.0 ml hot distilled water and squeezed into a separate beaker to collect the residual starch and washing discard was collected. This was repeated 3 times. AH the fractions were combined and the volume was made up to 100 mL with distilled water. 5 mL of this diluted extract was taken into 25 ml test tube and 10 mL freshly prepared anthrone reagent (2% in 95% H₂SO₄) was added. Tubes were placed in boiling water bath for 10 min, cooled to room temperature and A₅₄₀ was recorded. The glucose content was extrapolated from standard curve between glucose cone, and A₅₄₀. The value of glucose was multiplied by 0.9 to get starch content.

Determination of Residual Cellulose in Test Cloth after Washing

To determine the residual cellulose in washed cloth, it was dipped into 10 ml 5% H2SO4 for 2 hr at 90° C. in a water bath. The hydrolysis reaction was stopped by neutralizing the acid by adding small quantity of concentrated KOH solution. The glucose content in the hydrolyzed cellulose was determined by DNS reaction as described earlier. Glucose/cellulose content was extrapolated from standard curve between glucose cone, and A₅₄₀

Determination of Residual Casein in Test Cloths after Washing

To determine the residual protein content in the test cloth after washing, it was dipped into 10 ml (1N KOH) solution for 20 min under gentle shaking so that the residual casein of cloth got extracted into the solvent. Then the protein content in the solution was measured by Lowry's method.

### Determination of Residual Oil Content in Test Clothes

To determine the residual oil content of test cloth after washing, it was dipped into 10 ml of petroleum ether for 20 min with gentle shaking so that oil retained in test cloth was extracted into the fat solvent (petroleum ether). Then this fat solution was transferred to a 100 ml round bottom distillation flask. 25 ml of 0.5 M alcoholic potassium hydroxide was added to it. The flask was attached to a reflux condenser and the mixture was refluxed in a boiling water bath for 30 min. The flask was removed, cooled to room temperature and the mixture was titrated against 0.5 M HCl using 1% phenolphtheline as an indicator. The blank was set up similarly but no oil was taken in it. The volume of HCl consumed in the titration was noted.

TABLE 1
A comparison of kinetic parameter of free and co-immobilized α-amylase,
cellulose, protease and lipase on PVC/plastic container or brush

| | α-Amylase | | | Cellulase | | | | Protease | | Lipase | |
|---|---|---|---|---|---|---|---|---|---|---|---|
| Kinetic | | | Co- | | | Co- | | | Co- | | Co- |
| parameter | Free | | immobilized | Free | | immobilized | | Free | immobilized | Free | immobilized |
| Optimum | 4.5-5 | | 6 | 4.2 | | 5.8 | | 6.5 | 7.0 | 7.5 | 8 |
| pH | | | | | | | | | | | |
| Optimum | 55 | | 40 | 60 | | 40 | | 55 | 60 | 35 | 40 |
| temperature | | | | | | | | | | | |
| Time of | — | | 25 | — | | 35 | | 60 | 70 | 15 | 20 |
| incubation | | | | | | | | | | | |
| (min) | | | | | | | | | | | |
| Saturating | — | | 3M | — | | 0.2 | mM | 2M | 2M | 50 (mM) | 0.7M |
| conc. | | | | | | | | | | | |
| Kₘ | 1.73 | mg/ml | 1.25M | 0.05 | mM | 0.25 | mM | 8.3M | 6.25M | 4.242 × 10−3M | 1.25M |
| Vₘₐₓ | 1.67 | mg/min | 0.166 mg/ml/ | 6.6 | mg/ml/ | 0.83 | mg/ml/ | 0.47 mg/ml/min | 0.833M | 1.515 μMoles/min | 50 μMoles/min |
| | | | min | | min | | min | | mg/ml/min | | |

Commercial α-amylase from bacterial source, cellulose from Trichoderma viridae, protease from soybean (purified) and lipase from porcine pancreas were used.

TABLE 2
A comparison of washing performance (starch removal from
cotton cloth) of non enzymatic detergent with enzymatic detergent
in presence PVC beaker bound of co-immobilized α-amylase. The
values given in the table represent the residual content of starch
in cloth (mg/cm²) after washing.

| | Well | Canal | Ground | Distilled |
|---|---|---|---|---|
| Detergent Used | Water | Water | Water | Water |
| *Control | 0.866 | 0.884 | 0.693 | 0.768 |
| Non- Enzymatic | 0.642 | 0.431 | 0.631 | 0.551 |
| Enzymatic | 0.306 | 0.235 | 0.271 | 0.320 |
| Non enzymatic | 0.301 | 0.208 | 0.201 | 0.426 |
| Detergent + | | | | |
| Immobilized | | | | |
| Enzyme | | | | |

*Control: Contained no detergents but only water.
Ghari used as non enzymatic detergent
Surf Excel used as enzymatic detergent

TABLE 3
A comparison of washing performance (Grass stain removal from
cotton cloth) of non enzymatic detergent with enzymatic detergent
in presence PVC beaker bound of co-immobilized Cellulase. The
values in table represents residual content of cellulose
in cloth (mg/cm²)

| | Well | Canal | Ground | Distilled |
|---|---|---|---|---|
| Detergent Used | Water | Water | Water | Water |
| *Control | 0.084 | 0.884 | 0.693 | 0.768 |
| Non- Enzymatic | 0.071 | 0.431 | 0.631 | 0.426 |
| Enzymatic | 0.022 | 0.235 | 0.302 | 0.320 |
| Non enzymatic | 0.017 | 0.297 | 0.284 | 0.142 |
| Detergent + | | | | |
| Immobilized | | | | |
| Enzyme | | | | |

*Control: Contained no detergents but only water.
Ghari used as non enzymatic detergent
Surf Excel used as enzymatic detergent

TABLE 4
A comparison of washing performance (Egg albumin stain removal
from cotton cloth) of non enzymatic detergent with enzymatic
detergent in presence of PVC beaker bound co-immobilized
Protease. The values given in table represent the residual
content of egg albumin after washing (mg/cm2). Lesser the
albumin content betters the washing.

| | Well | Canal | Ground | Distilled |
|---|---|---|---|---|
| Detergent Used | Water | Water | Water | Water |
| *Control | 1.220 | 0.854 | 1.697 | 1.831 |
| Non- Enzymatic | 0.600 | 1.244 | 0.906 | 1.104 |
| Enzymatic | 0.531 | 0.743 | 0.542 | 0.885 |
| Non- Enzymatic | 0.482 | 0.690 | 0.671 | 0.760 |
| Detergent + | | | | |
| Immobilized | | | | |
| Enzyme | | | | |

*Control: Contained no detergents but only water.
Ghari used as non enzymatic detergent
Surf Excel used as enzymatic detergent

TABLE 5
A comparison of washing performance (oil stain removal from
cotton cloth) of non enzymatic detergent with enzymatic detergent
in presence of PVC beaker bound co-immobilized Lipase. The values
given in the table represent the residual content of oil (μmole
equivalent/cm²) after washing.

| | Well | Canal | Ground | Distilled |
|---|---|---|---|---|
| Detergent Used | Water | Water | Water | Water |
| *Control | 28.7 | 31.25 | 36.25 | 28.75 |
| Non- Enzymatic | 18.75 | 15.0 | 18.75 | 16.25 |
| Enzymatic | 12.5 | 7.5 | 13.75 | 8.25 |
| Non- Enzymatic | 12.5 | 8.75 | 12.5 | 6.25 |
| Detergent + | | | | |
| Immobilized | | | | |
| Enzyme | | | | |

*Control: Contained no detergents but only water.
Ghari used as non enzymatic detergent.
Surf Excel used as enzymatic detergent.

TABLE 6
A comparison of washing performance (starch removal from
cotton cloth) of non enzymatic detergent with enzymatic
detergent in presence of PVC brush bound co-immobilized α-amylase.
The values given in the table represent the residual content of
starch in cloth (mg/cm²) after washing.

| | Well | Canal | Ground | Distilled |
|---|---|---|---|---|
| Detergent Used | Water | Water | Water | Water |
| *Control | 1.788 | 1.404 | 0.983 | 1.764 |
| Non- Enzymatic | 0.942 | 0.971 | 0.562 | 0.551 |
| Enzymatic | 0.686 | 0.590 | 0.471 | 0.434 |
| Non enzymatic | 0.402 | 0.735 | 0.220 | 0.327 |
| Detergent + | | | | |
| Immobilized | | | | |
| Enzyme | | | | |

*Control: Contained no detergents but only water.
Ghari used as non enzymatic detergent
Surf Excel used as enzymatic detergent

TABLE 7
A comparison of washing performance (Grass stain removal from
cotton cloth) of non enzymatic detergent with enzymatic detergent
in presence of PVC brush bound co-immobilized Cellulase. The
values in table represents residual content of cellulose
in cloth (mg/cm²)

| | Well | Canal | Ground | Distilled |
|---|---|---|---|---|
| Detergent Used | Water | Water | Water | Water |
| *Control | 0.964 | 0.884 | 0.834 | 0.796 |
| Non- Enzymatic | 0.867 | 0.721 | 0.609 | 0.602 |
| Enzymatic | 0.532 | 0.468 | 0.576 | 0.553 |
| Non enzymatic | 0.369 | 0.405 | 0.512 | 0.328 |
| Detergent + | | | | |
| Immobilized | | | | |
| Enzyme | | | | |

*Control: Contained no detergents but only water.
Ghari used as non enzymatic detergent
Surf Excel used as enzymatic detergent

TABLE 8
A comparison of washing performance (Egg albumin stain removal
from cotton cloth) of non enzymatic detergent with enzymatic
detergent in presence of PVC brush bound co-immobilized
Protease. The values given in table represent the residual
content of egg albumin after washing (mg/cm2). Lesser the
albumin content betters the washing.

| | Well | Canal | Ground | Distilled |
|---|---|---|---|---|
| Detergent Used | Water | Water | Water | Water |
| *Control | 1.067 | 0.970 | 0.982 | 1.651 |
| Non- Enzymatic | 0.932 | 0.901 | 0.872 | 1.044 |
| Enzymatic | 0.631 | 0.932 | 0.644 | 0.905 |
| Non- Enzymatic | 0.442 | 0.784 | 0.433 | 0.658 |
| Detergent + | | | | |
| Immobilized | | | | |
| Enzyme | | | | |

*Control: Contained no detergents but only water.
Ghari used as non enzymatic detergent
Surf Excel used as enzymatic detergent

TABLE 9
A comparison of washing performance (oil stain removal from
cotton cloth) of non enzymatic detergent with enzymatic detergent
in presence of PVC beaker bound co-immobilized Lipase. The values
given in the table represent the residual content of oil (μmole
equivalent/cm²) after washing.

| | Well | Canal | Ground | Distilled |
|---|---|---|---|---|
| Detergent Used | Water | Water | Water | Water |
| *Control | 20.7 | 22.62 | 26.22 | 21.32 |
| Non- Enzymatic | 19.44 | 17.1 | 22.07 | 18.05 |
| Enzymatic | 17.8 | 8.5 | 17.3 | 12.15 |
| Non- Enzymatic | 16.2 | 8.8 | 14.29 | 8.33 |
| Detergent + | | | | |
| Immobilized | | | | |
| Enzyme | | | | |

*Control: Contained no detergents but only water.
Ghari used as non enzymatic detergent.
Surf Excel used as enzymatic detergent.

I/We claim:

1. A polyvinyl chloride surface for removing stains, wherein the polyvinyl chloride surface is co-immobilized with enzymes lipase, cellulase, amylase and protease.

2. The polyvinyl chloride surface as claimed in claim 1, wherein the surface is in the form of sheet, brush, vessel, pipe, reactor, chips, disc, strip and gauge.

3. The polyvinyl chloride surface as claimed in claim 1 is capable of removing stains in distilled water, canal water, ground water, tap water, well water and hard water.

4. The polyvinyl chloride surface as claimed in claim 1 is capable of removing stains in presence of non-enzymatic detergents.

5. The polyvinyl chloride surface as claimed in claim 1 is capable of removing starch, protein, grass, oil, soil, blood, grease, sauces, ice-creams, gravies, egg, human sweat, chocolate, dust and mud.

6. The polyvinyl chloride surface as claimed in claim 1 is reusable for at least 200 times.

7. The polyvinyl chloride surface as claimed in claim 1 is polyvinyl chloride vessel or brush.
